(19) United States
(12) Patent Application Publication
(10) Pub. No.: US 2022/0185749 A1
(43) Pub. Date: Jun. 16, 2022

(54) LEWIS ACID CATALYSED SYNTHESIS OF 1,2-BIS(PERFLUOROALKYL)ETHYLENES

(71) Applicant: THE CHEMOURS COMPANY FC, LLC, WILMINGTON, DE (US)

(72) Inventors: VIACHESLAV A PETROV, HOCKESSIN, DE (US); ROBERT DANIEL LOUSENBERG, WILMINGTON, DE (US)

(73) Assignee: THE CHEMOURS COMPANY FC, LLC, WILMINGTON, DE (US)

(21) Appl. No.: 17/442,641

(22) PCT Filed: Apr. 17, 2020
(86) PCT No.: PCT/US2020/028687

§ 371 Date: Sep. 24, 2021

Related U.S. Application Data
(60) Provisional application No. 62/835,714, filed on Apr. 18, 2019.

(51) Int. Cl. C07C 17/278

(52) U.S. Cl. CPC C07C 17/278

(57) ABSTRACT

A method of producing a fluoroolefin includes contacting a compound of formula (1), R_{f}CH═CHF, with a fluorinated ethylene compound of formula (2), CX₁X₂═CX₃X₄ in the presence of a Lewis acid catalyst. In the compound of formula (1), R_{f} is a C₁-C₁₀ perfluorinated alkyl group. In the compound of formula (2), X₁, X₂, X₃, and X₄ are each independently H, Cl, or F and at least one of X₁, X₂, X₃, and X₄ is F. The resulting composition comprises a compound of formula (3), R_{f}CF₃(CX₅X₆CX₇X₈)ₙCH═CHCX₉X₁₀CX₁₁X₁₂F. In the compound of formula (3), X₅, X₆, X₇, X₈, X₉, X₁₀, X₁₁, and X₁₂ are each independently H, Cl, or F, n is an integer of 0 or 1, and the total number of each of H, Cl, and F corresponds to the total number of each of H, Cl, and F provided by the fluorinated ethylene compound of formula (2).

This application claims the benefit of Application No. 62/835,714, filed Apr. 18, 2019. The disclosure of Application No. 62/835,714 is hereby incorporated by reference.

### FIELD

The present invention is directed to the production of fluorinated alkene compounds.

### BACKGROUND

The is interest in low temperature heat utilization (i.e., heat at temperatures lower than about 300° C.). Such heat may be extracted from various commercial, industrial or natural sources. Elevation of the temperature of available heat through high temperature mechanical compression heat pumps (HTHPs) to meet heating requirements and conversion of the available heat to mechanical or electrical power through Organic Rankine Cycles (ORCs) are two promising approaches for the utilization of low temperature heat.

ORCs and HTHPs require the use of working fluids. Working fluids with high global warming potentials (GWPs) currently in common use for HTHPs and ORCs (e.g. HFC-245fa) have been under review and there is a need for more environmentally sustainable working fluids for HTHPs and ORCs. More specifically, there is a need for low GWP working fluids with boiling points higher than about 50° C. that are particularly suitable for conversion of heat available at temperatures approaching or exceeding 200 degrees Celsius (hereinafter “° C.”) to power and for heating at temperatures approaching 200° C. from heat available at lower temperatures. Even more specifically, a low GWP working fluid with a boiling point close to that of ethanol (78.4° C.) could be advantageous as a replacement of ethanol in ORC systems for heavy duty vehicles (e.g., trucks) especially in Europe. Such a fluid could also be used as a solvent and as a heat transfer fluid for various applications, including immersion cooling and phase change cooling (e.g., of electronics, including data center cooling).

Fluoroalkenes, such as, F23E (C₂F₅CH═CHC₃F₇) can be prepared using F-heptene-3 starting material using a four-step preparation, including sequential hydrogenation/dehydrofluorination process. However, this process is lengthy and is based on relatively expensive starting materials (F-heptene is made using the reaction of hexafluoropropene (HFP) and 2 moles of tetrafluoroethene (TFE)).

WO 2008/057513 describes a process for the preparation of internal dihydrofluoroolefins of the formula RCH═CHC₂F₅, comprising reacting a fluorinated olefin of the RCH═CHF, wherein R is selected from perfluoroalkyl groups having from one to ten carbon atoms, and the said alkyl group is either an n-alkyl chain, a sec-alkyl chain, or an iso-alkyl chain, in the liquid phase with tetrafluoroethylene, in the presence of an antimony pentafluoride (SbF₅), removing the Lewis acid catalyst and isolating the dihydrofluoroolefin. The disclosure of WO 2008/057513 is hereby incorporated by reference.

### SUMMARY

One embodiment of the invention relates to a method of producing a fluoroolefin comprising:
contacting a compound of formula (1),

R_{f}CH═CHF (1)
wherein R_{f} is a C₁-C₁₀ perfluorinated or polyfluorinated alkyl group; with a fluorinated ethylene compound of formula (2),

CX₁X₂═CX₃X₄ (2)
wherein X₁, X₂, X₃, and X₄ are each independently H, Cl, or F; and wherein at least one of X₁, X₂, X₃, and X₄ is F;
in the presence of a Lewis acid catalyst in an amount sufficient to form a composition comprising a compound of formula (3),

R_{f}CF₃(CX₅X₆CX₇X₈)ₙCH═CHCX₉X₁₀CX₁₁X₁₂F (3)
wherein X₅, X₆, X₇, X₈, X₉, X₁₀, X₁₁, and X₁₂ are each independently H, Cl, or F, n is an integer of 0 or 1; and
wherein the total number of each of H, Cl, and F represented by X₅, X₆, X₇, X₈, X₉, X₁₀, X₁₁, and X₁₂ is the same as the total number of each of H, Cl, and F provided by the fluorinated ethylene compound of formula (2).

Another embodiment of the invention comprises the foregoing embodiment wherein:
the compound of formula (1) includes CF₃CH═CHF (1234ze); and
the composition includes 1,1,1,4,4,5,5,5-octafluoropent-2-ene, CF₃CH═CHC₂F₅ (F12E).

Another embodiment of the invention comprises any combination of the foregoing embodiments wherein:
the fluorinated ethylene of formula (2) includes CF₂═CF₂ (TFE); and
the composition includes 1,1,1,4,4,5,5,5-octafluoropent-2-ene, CF₃CH═CHC₂F₅ (F12E).

Another embodiment of the invention comprises any combination of the foregoing embodiments wherein:
the compound of formula (1) includes CF₃CH═CHF (1234ze); and
the fluorinated ethylene of formula (2) includes CF₂═CF₂ (TFE); and
the composition includes 1,1,1,4,4,5,5,5-octafluoropent-2-ene, CF₃CH═CHC₂F₅ (F12E).

Another embodiment of the invention comprises any combination of the foregoing embodiments wherein the amount sufficient includes a molar ratio of (TFE):(1234ze) of 0.01:1 to 5:1.

Another embodiment of the invention comprises any combination of the foregoing embodiments wherein the amount sufficient includes a molar ratio of (TFE):(1234ze) of 0.1:1 to 2:1.

Another embodiment of the invention comprises any combination of the foregoing embodiments wherein the amount sufficient includes a molar ration of the compound of formula (2) and the compound of formula (1) of 0.01:1 to 5:1.

Another embodiment of the invention comprises any combination of the foregoing embodiments wherein the composition further comprises 1,1,1,2,2,5,5,6,6,7,7,7-dodecafluorohept-2-ene, C₃F₇CH═CHC₂F₅ (F23E).

Another embodiment of the invention comprises any combination of the foregoing embodiments wherein the composition further comprises 4-chloro-1,1,1,4,5,5,5-heptafluoropent-2-ene CF₃CH═CHCFClCF₃ and 5-chloro-1,1,1,4,4,5,5-heptafluoropent-2-ene CF₃CH═CHCF₂CF₂Cl.

Another embodiment of the invention comprises any combination of the foregoing embodiments wherein the composition further includes at least one of diluents and solvents.

Another embodiment of the invention comprises any combination of the foregoing embodiments wherein the solvent is a perfluorinated saturated compound.

Another embodiment of the invention comprises any combination of the foregoing embodiments wherein the perfluorinated saturated compound is selected from the group insisting of perfluoropentane, perfluorohexane, cyclic dimer of hexafluoropropene, (mixture of perfluoro-1,2- and perfluoro-1,3-dimethylcyclobutanes), and combinations thereof

Another embodiment of the invention comprises any combination of the foregoing embodiments wherein:
the fluorinated ethylene of formula (2) includes CClF═CF₂ (CTFE); and
the composition includes 4-chloro-1,1,1,4,5,5,5-heptafluoropent-2-ene, CF₃CH═CHCClFCF₃ or 5-chloro-1,1,1,4,4,5,5-heptafluoropent-2-ene, CF₃CH═CHCF₂CClF₂.

Another embodiment of the invention comprises any combination of the foregoing embodiments wherein:
the catalyst includes aluminum chloride (AlCl₃) or a compound of formula (4),

AlClₓF₃₋ₓ
wherein x=0.01 to 0.5.

Another embodiment of the invention comprises any combination of the foregoing embodiments wherein the catalyst includes aluminum chloride (AlCl₃).

Another embodiment of the invention comprises any combination of the foregoing embodiments wherein R_{f} is a C₂-C₁₀ perfluorinated alkyl group.

Another embodiment of the invention comprises any combination of the foregoing embodiments wherein R_{f} is CF₃, C₂F₅, C₃F₇, iC₃F₇, C₄F₉, C₅F₁₁, i-C₅F₁₁,

C₆F₁₃ or i-C₆F₁₃.

Another embodiment of the invention comprises any combination of the foregoing embodiments wherein the contacting is performed at sub-ambient or ambient temperature.

Another embodiment of the invention comprises any combination of the foregoing embodiments wherein the reaction is conducted under autogenic pressure.

Another embodiment of the invention comprises any combination of the foregoing embodiments wherein the reaction is conducted at 0.1 to 300 psig.

Another embodiment of the invention comprises any combination of the foregoing embodiments wherein the reaction is conducted in a closed system.

Another embodiment of the invention comprises any combination of the foregoing embodiments wherein the contacting is performed at a temperature of −50° C. to 50° C.

Another embodiment of the invention comprises any combination of the foregoing embodiments wherein the catalyst includes aluminum chloride (AlCl₃) or aluminum chlorofluoride AlClₓF₃₋ₓ (ACF) wherein x=0.01 to 0.5.

Another embodiment of the invention comprises any combination of the foregoing embodiments wherein the catalyst includes aluminum chloride (AlCl₃).

Another embodiment of the invention comprises any combination of the foregoing embodiments wherein the at least of diluents and solvents comprises a reaction product formed by said contacting.

One embodiment of the invention relates to a method of producing a fluoroolefin comprising:

contacting CF₃CH═CHF (1234ze) with CF₂═CF₂ (TFE) in the presence of a catalyst in an amount sufficient to form a composition comprising 1,1,1,2,2,5,5,6,6,7,7,7-dodecafluorohept-2-ene, C₃F₇CH═CHC₂F₅ (F23E).

Another embodiment of the invention comprises any combination of the foregoing embodiments wherein the contacting is performed at a temperature of −10° C. to 50° C.

Another embodiment of the invention comprises any combination of the foregoing embodiments wherein the composition further comprises 1,1,1,4,4,5,5,5-octafluoropent-2-ene, CF₃CH═CHC₂F₅ (F12E).

Another embodiment of the invention comprises any combination of the foregoing embodiments wherein the composition further comprises 4-chloro-1,1,1,4,5,5,5-heptafluoropent-2-ene, CF₃CH═CHCFClCF₃ and 5-chloro-1,1,1,4,4,5,5-heptafluoropent-2-ene, CF₃CH═CHCF₂CF₂C₁.

Another embodiment of the invention comprises any combination of the foregoing embodiments wherein the composition further includes at least one of diluents and solvents.

Another embodiment of the invention comprises any combination of the foregoing embodiments wherein the solvent is a perfluorinated saturated compound.

Another embodiment of the invention comprises any combination of the foregoing embodiments wherein the perfluorinated saturated compound is selected from the group insisting of perfluoropentane, perfluorohexane, cyclic dimer of hexafluoropropene, (mixture of perfluoro-1,2- and perfluoro-1,3-dimethylcyclobutanes), and combinations thereof

Another embodiment of the invention comprises any combination of the foregoing embodiments wherein the amount sufficient includes a molar ratio of (TFE):(1234ze) of 0.01:1 to 5:1.

Another embodiment of the invention comprises any combination of the foregoing embodiments wherein the amount sufficient includes a molar ratio of (TFE):(1234ze) of 0.1:1 to 2:1.

Another embodiment of the invention comprises any combination of the foregoing embodiments wherein the contacting is performed at sub-ambient or ambient temperature.

Another embodiment of the invention comprises any combination of the foregoing embodiments wherein the reaction is conducted under autogenic pressure.

Another embodiment of the invention comprises any combination of the foregoing embodiments wherein the reaction is conducted at 0.1 to 300 psig.

Another embodiment of the invention comprises any combination of the foregoing embodiments wherein the reaction is conducted in a closed system.

Another embodiment of the invention comprises any combination of the foregoing embodiments wherein the catalyst includes aluminum chloride (AlCl₃).

Another embodiment of the invention comprises any combination of the foregoing embodiments wherein the at least one diluent and solvent comprises a reaction product formed by said contacting.

One embodiment of the invention relates to a method of producing a fluorooligomer comprising:

heating CF₃CH═CHF (1234ze), in the presence of a catalyst, at a temperature and a pressure sufficient to form a composition comprising CF₃CH═CHCH(CF₃)CF₂H.

Another embodiment of the invention comprises any combination of the foregoing embodiments wherein the catalyst includes aluminum chloride (AlCl₃) or aluminum chlorofluoride AlC₁ₓF₃₋ₓ (ACF) wherein x=0.01 to 0.5.

In an embodiment, a method of producing a fluoroolefin includes contacting a compound of formula (1), R_{f}CH═CHF, with a fluorinated ethylene compound of formula (2), CX₁X₂═CX₃X₄. In the compound of formula (1) R_{f} is a C₁-C₁₀ perfluorinated alkyl group. In the compound of formula (2), X₁, X₂, X₃, and X₄ are each independently H, Cl, or F and at least one of X₁, X₂, X₃, and X₄ is F. The contacting is performed in the presence of a Lewis acid catalyst in an amount and under conditions sufficient to form a composition comprising a compound of formula (3), R_{f}CF₃(CX₅X₆CX₇X₈)ₙCH═CHCX₉X₁₀CX₁₁X₁₂F. In the compound of formula (3) X₅, X₆, X₇, X₈, X₉, X₁₀, X₁₁, and X₁₂ are each independently H, Cl, or F and the total number of each of H, Cl, and F represented by X₅, X₆, X₇, X₈, X₉, X₁₀, X₁₁, and X₁₂ is the same as the total number of each of H, Cl, and F provided by the fluorinated ethylene compound of formula (2).

In another embodiment, a method of producing a fluoroolefin includes contacting CF₃CH═CHF (1234ze) with CF₂═CF₂ (TFE) in the presence of a catalyst in an amount sufficient to form a composition comprising 1,1,1,2,2,5,5,6,6,7,7,7-dodecafluorohept-2-ene, C₃F₇CH═CHC₂F₅ (F23E).

In another embodiment, a method of producing a fluorooligomer includes heating CF₃CH═CHF (1234ze), in the presence of a catalyst, at a temperature and a pressure sufficient to form a composition comprising CF₃CH═CHCH(CF₃)CF₂H.

One embodiment of the invention relates to a composition formed by any combination of the foregoing methods.

The embodiments can be used alone or in combinations with each other.

Other features and advantages of the present invention will be apparent from the following more detailed description, taken in conjunction with the accompanying drawings which illustrate, by way of example, the principles of the invention.

### DETAILED DESCRIPTION

Provided is a one-step synthesis for the production of fluorinated alkenes.

Embodiments of the present disclosure, for example, in comparison to concepts failing to include one or more of the features disclosed herein, provide a one-step synthesis for the production of fluorinated alkenes. More specifically, the present disclosure provides a one-step synthesis for the production of fluorinated alkenes having a perfluorinated alkyl chain.

The process may be conducted in any reactor suitable for a vapor phase fluorination reaction. The reactor is made of a material that is resistant to the reactants employed. The reactor may be constructed from materials which are resistant to the corrosive effects of hydrogen fluoride such as stainless steel, Hastelloy®, Inconel®, Monel®, gold or gold-lined or quartz. The reactions may be conducted batchwise, continuous, semi-continuous or combinations thereof. Suitable reactors include batch reactor vessels and tubular reactors.

In an embodiment a compound of formula (1),

R_{f}CH═CHF (1)
wherein R_{f} is a C₁-C₁₀ perfluorinated alkyl group;
is charged to a reactor, heated, and contacted, in the presence of a catalyst, with a fluorinated ethylene compound of formula (2),

CX₁X₂═CX₃X₄ (2)
wherein X₁, X₂, X₃, and X₄ are each independently H, Cl, or F; and
wherein at least one of X₁, X₂, X₃, and X₄ is F.

The temperature and pressure of the reactor are maintained at levels sufficient to effect, in the presence of a Lewis acid catalyst, the formation of a composition comprising a compound of formula (3),

R_{f}CF₃(CX₅X₆CX₇X₈)ₙCH═CHCX₉X₁₀CX₁₁X₁₂F (3)

wherein X₅, X₆, X₇, X₈, X₉, X₁₀, X₁₁, and X₁₂ are each independently H, Cl, or F, n is an integer of 0 or 1; and

wherein the total number of each of H, Cl, and F represented by X₅, X₆, X₇, X₈, X₉, X₁₀, X₁₁, and X₁₂ is the same as the total number of each of H, Cl, and F provided by the fluorinated ethylene compound of formula (2).

In some embodiments, the compound of formula (1) includes 1,3,3,3-tetrafluoro-1-propene, CF₃CH═CHF (1234ze). In one embodiment, the compound of formula (1) includes CF₃CH═CHF (1234ze). In some embodiments, R_{f} may be a linear or branched perfluorinated or polyfluorinated alkyl group. In some embodiments, R_{f} may be CF₃, C₂F₅, C₃F₇, iC₃F₇, C₄F₉, C₅F₁₁, i-C₅F₁₁, C₆F₁₃ or i-C₆F₁₃.

In some embodiments, the fluorinated ethylene of formula (2) includes at least one of tetrafluoro-ethene, CF₂═CF₂ (TFE) or CFC1═CF₂ (1-chloro-1,2,2-trifluoro-ethene), CF₂═CH₂ (1,1-difluoro-ethene), CH₂═CHF (1-fluoro-ethene), CF₂═CCl₂ (1,1-dichloro-2,2-difluoro-ethene), CFC1═CFC1 (1,2-chloro-1,2-difluoro-ethene). In one embodiment, the fluorinated ethylene of formula (2) includes tetrafluoro-ethene, CF₂═CF₂ (TFE).

In one embodiment, the compound of formula (1) includes CF₃CH═CHF (1234ze) and the fluorinated ethylene of formula (2) includes CF₂═CF₂ (TFE). The reaction of CF₃CH═CHF (1234ze) and CF₂═CF₂ (TFE) may result in the formation of the composition including 1,1,1,4,4,5,5,5-octafluoropent-2-ene, CF₃CH═CHC₂F₅ (F12E).

If desired, the 1,1,1,4,4,5,5,5-octafluoropent-2-ene may be isolated and optionally purified prior to use. Suitable uses of 1,1,1,4,4,5,5,5-octafluoropent-2-ene include, but are not limited to, working fluids in systems utilizing a thermodynamic cyclic, a reactive intermediate, a refrigerant, a heat transfer fluid with or without phase change, and a solvent.

In some embodiments, fluorinated ethylene of formula (2) may include a plurality of compounds of formula (2). The resulting compound of formula (3) may include a plurality of compounds of formula (3). In one embodiment, the fluorinated ethylene of formula (2) may include tetrafluoro-ethene, CF₂═CF₂ (TFE) and 1-chloro-1,2,2-trifluoro-ethene. In a further embodiment, the compound of formula (1) may include CF₃CH═CHF (1234ze).

The resulting compound of formula (3) may include 1,1,1,4,4,5,5,5-octafluoropent-2-ene, CF₃CH═CHC₂F₅ (F12E), and 4-chloro-1,1,1,4,5,5,5-heptafluoropent-2-ene CF₃CH═CHCFClCF₃ and/or 5-chloro-1,1,1,4,4,5,5-heptafluoropent-2-ene CF₃CH═CHCF₂CF₂Cl, 4,5-dichloro-1,1,1,4,5,5-hexafluoropent-2-ene, CF₃CH═CHCFClCF₂Cl, 1,1,1,5,5,5-hexafluoropent-2-ene CF₃CH═CHCH₂CF₃. In an alternate embodiment, the compound of formula (3) may include 4-chloro-1,1,1,4,5,5,5-heptafluoropent-2-ene, CF₃CH═CHCClFCF₃ or 5-chloro-1,1,1,4,4,5,5-heptafluoropent-2-ene, CF₃CH═CHCF₂CClF₂.

The molar ratio of a formula (2) compound to a formula (1) compound, which are contacted in accordance with the invention, can be used be control the composition and ratio of reaction products. In some embodiments, the compound of formula (2) and the compound of formula (1) are contacted in amounts resulting in a molar ratio of 0.01:1 to 5:1. In one embodiment, the compound of formula (2) and the compound of formula (1) are contacted in amounts resulting in a molar ratio of (2):(1) of 0.1:1 to 2:1. A contact molar ratio of about 1:1 can produce C₅ compounds and a molar ratio of about 2:1 can product C₇ compounds. While any desired ratio can be employed, a ratio of about 2:1 is useful. In one embodiment, the compound of formula (2) and the compound of formula (1) are contacted in amounts resulting in a molar ratio of (2):(1) of 1:1 to 2:1. In an embodiment, the compound of formula (2) is (TFE) and the compound of formula (1) is (1234ze).

The reaction conditions and stoichiometry may be selected to allow the compound of formula (3) such as, the 1,1,1,4,4,5,5,5-octafluoropent-2-ene, CF₃CH═CHC₂F₅ (F12E) described above, to act as a reactive intermediate. In some embodiments, the fluorinated ethylene of formula (2) may be provided in a stoichiometric excess with respect to the amount of the compound of formula (1). In some embodiments, the excess of the compound of formula (2), such as (TFE), allows one or more additional units of the compound of formula (2) to react with the 1,1,1,4,4,5,5,5-octafluoropent-2-ene to form additional compounds of formula (3), having an extended carbon chain. In one embodiment, the composition comprising the compound of formula (3) may include 1,1,1,2,2,5,5,6,6,7,7,7-dodecafluorohept-2-ene, C₃F₇CH═CHC₂F₅ (F23E).

The reaction is typically conducted in a closed system. In some embodiments, the Lewis acid is a strong Lewis acid. In one embodiment, the catalyst is, aluminum chloride (AlCl₃), or antimony pentafluoride (SbF₅), or aluminum chlorofluoride AlClₓF₃₋ₓ. In some embodiments, for aluminum based catalyst x may be an integer from 1 to 3. In some embodiments, x may be 0.01 to 0.5. The amount of catalyst can range from about 0.1 to about 20 weight percent of the reaction mixture, in some cases about 1 to about 15 and in some cases about 5 to about 10 wt. %.

Additional suitable strong Lewis acids may be found in (Chemical Reviews, 1996, v. 96, pp. 3269-3301; a list of strong Lewis acids is given on page 3271), which is hereby incorporated by reference. In some embodiments, the reaction mixture is heated to a sub-ambient or ambient temperature. In some embodiments, the reaction mixture is heated to a temperature of −50° C. to 50° C. In one embodiment, the reaction mixture is heated to a temperature of −50° C. to 25° C. In some embodiments, the reaction is performed at a reactor pressure of 0.1 pound per square inch gauged (psig) to 300 pounds per square inch gauged (psig). In some embodiments, the reaction is performed under autogenic pressure.

In some embodiments, the formation of the compound of formula (3) may be conducted in the presence of at least one of a solvent or a diluent; depending upon whether all components of a reaction mixture are soluble In some embodiments, the solvent or diluent is a perfluorinated saturated compound. In some embodiments, the perfluorinated saturated compound may include perfluoropentane, perfluorohexane, cyclic dimer of hexafluoropropene, (mixture of perfluoro-1,2- and perfluoro-1,3-dimethylcyclobutanes), and combinations thereof or the product of the reaction can be used as a reaction media The amount of at least one solvent or diluent can range from about 10 to about 50 volume percent of the reaction vessel, about 15 to 40 and in some cases about 20 to 30 volume percent.

In one specific embodiment, the at least one diluent or solvent comprises a reaction product formed by contacting formulas (1) and (2). The reaction product diluent or solvent can be supplied to a reaction environment by recycling a portion of a recovered reaction product in a continuous method, leaving a residual portion of the reaction product in the reaction environment in a batch method, among other suitable techniques for delivering a diluent or solvent to a reaction environment.

Compounds of formula (3) may be used in numerous applications for the transfer of heat, such as, heat transfer fluids or refrigerants. In one embodiment, the compounds of formula (3) may be used to transfer heat from an article. The article may be contacted with a heat transfer media including at least one compound of formula (3).

In an alternate embodiment, the compound of formula (1) may be dimerized. The compound of formula (1) may be reacted with itself, in the absence of the fluorinated ethylene compound of formula (2), in the presence of a catalyst, such as antimony fluoride (SbF₅). In some embodiments, the reaction may be performed in the presence of a solvent. Suitable solvents include those described above.

In an example of the alternate embodiment, a dimer may be formed by reacting 1,3,3,3-tetrafluoro-1-propene, CF₃CH═CHF (1234ze), as shown below.

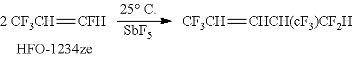

In one embodiment of the invention, the reaction is conducted in an environment that is free or substantially free of compounds having OH groups. Examples of such OH containing compounds are hydrocarbon grease or oil, and solvents with OH group such as water or alcohol. By substantially free, it is meant that less than 50 ppm, less than 25 ppm and in some cases less than 10 ppm of OH containing compounds are present.

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method that includes materials, steps, features, components, or elements, in addition to those literally disclosed provided that these additional included materials, steps, features, components, or elements do materially affect the basic and novel characteristic(s) of the claimed invention, especially the mode of action to achieve the desired result of any of the processes of the present invention. The term ‘consisting essentially of’ occupies a middle ground between “comprising” and ‘consisting of’.

Where applicants have defined an invention or a portion thereof with an open-ended term such as “comprising,” it should be readily understood that (unless otherwise stated) the description should be interpreted to also include such an invention using the terms “consisting essentially of” or “consisting of.”

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

The following Examples are provided to illustrate certain embodiments of the invention and shall not limit the scope of any claims appended hereto.

### EXAMPLES

Exemplary examples of the formation of compounds of formula (3) are shown below:

### Example 1

Reaction of HFO-1234ze with chlorotrifluoroethylene catalyzed by SbF₅

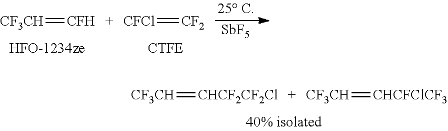

A 400 ml Hastelloy® shaker tube was loaded with 12 g (0.055 mol) of SbF₅, shaker tube was cooled down in dry ice, evacuated and charged with 150 g (1.32 mol) of HFO-1234ze and 150 g (1.29 mol) of chlorotrifluoroethylene (CTFE). It was placed in barricade and was warmed up to ambient temperature and kept agitated for 16 housr. The reaction vessel was cooled down with ice, vented off and liquid product was added to 1 L of water. Organic layer was separated, dried over MgSO₄ and filtered to give 290 g of crude material. It was fractionated to give 148 g (50% yield) of fraction b.p. 59-62° C., which was identified as a mixture of CF₃CH═CHCF₂CF₂C₁ and CF₃CH═CHCFClCF₃ in ratio 36:64 (purity of this fraction was 97.8%). This fraction was redistilled to give 120 g of material 99.3% purity, b.p. 60-61° C.

E-CF₃CH═CHCF₂CF₂Cl:

¹⁹F NMR (CDCl₃): −66.38 (3F, m), −71.74 (2F, m), −113.98 (2F, m) ppm

E-CF₃CH═CHCFClCF₃:

¹⁹F NMR (CDCl₃): −66.90 (3F, m), −82.15 (3F, m), −131.82 (1F, m) ppm

¹H NMR(CDCl₃, mixture isomers): 6.48 (m) ppm

GC/MS (m/z, mixture of isomers): 230 (M⁺, C₅H₂ClF₇⁺)

The ratio of CF₃CH═CHCF₂CF₂C₁ and CF₃CH═CHCFClCF₃ in the reaction product mixture can vary. The reaction product ratio can range from about 30:70, about 32:68, about 34:66 and in some cases about 36:64.

### Example 2

Reaction of HFO-1234ze with chlorotrifluoroethylene catalyzed by AlCl₃

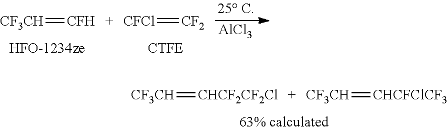

A 400 ml Hastelloy® shaker tube was loaded with 12 g (0.09 mol) of anhydrous pulverized AlCl₃. The shaker tube was cooled down in dry ice, evacuated and charged with 75 g (0.66 mol) of HFO-1234ze and 75 g (0.64 mol) of chlorotrifluoroethylene (CTFE). The shaker tube was placed in barricade, warmed up to ambient temperature and kept agitated for 16 hours. The reactor was cooled down with ice, vented off and liquid product was added to 1 L of water. Organic layer was separated, dried over MgSO₄ and filtered to give 148 g of crude material, which was found to contain 68% of a mixture of CF₃CH═CHCF₂CF₂C₁ and CF₃CH═CHCFClCF₃ (ratio of CF₃CH═CHCF₂CF₂C₁ and CF₃CH═CHCFClCF₃ 54:46) along with higher boiling point materials. The calculated yield of the C₅H₂ClF₇ fraction was 66%.

If desired, the amount of catalyst can be varied. The reaction product ratio of CF₃CH═CHCF₂CF₂C₁ and CF₃CH═CHCFClCF₃ can range from about 64:36, about 62:38, and in some cases about 60:40.

Reaction of HFO-1234ze with SbF₅ (Comparative Example).

A 1 L Hastelloy® agitated reactor was charged with 11 g (0.05 mol) of SbF₅, cooled down with dry ice, leak checked by pressurizing with nitrogen, vented, evacuated and 500 g (4.4 mol) of HFO1234ze was condensed into the reactor. It was brought to ambient temperature and kept at 25-30° C. for 12 hours Water (100 ml) was injected into the reactor using a pump. The reactor was vented, opened and the reaction mixture was added to separatory funnel containing 1 L of water, organic layer was separated, dried oven MgSO₄, filtered to give 474 g of crude product, which was further flash distilled to yield 400 g of crude product. Fractionation using 36 inch glass column with Hastelloy® packing and gave 350 g (70% yield) of material with b.p. 86-87° C., identified by NMR and GC/MS as E-CF₃CH═CHCH(CF₃)CF₂H, containing 3% of Z-isomer.

E-CF₃CH═CHCH(CF₃)CF₂H:

¹⁹F NMR (CDCl₃): −65.86 (3F, m), −67.47 (3F, m), −120.00 (1F, ddm, 300, 54.1 Hz), −123.60 (1F, ddm, 300, 54.1 Hz) ppm

¹H (NMR(CDCl₃, mixture isomers): 6.06 (1H, m), 6.10 (1H, t, d, 54.1, 2.5 Hz), 6.33 (1H, m) ppm

GC/MS (m/z): 228 (M⁺, C₆H₄F₈⁺)

### Example 3

Reaction of HFO-153-10ze with chlorotrifluoroethylene catalyzed by AlCl₃

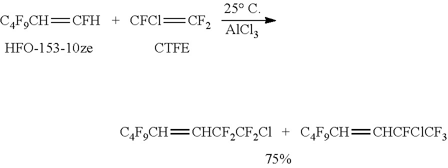

A 50 ml flask was loaded with 1.0 g (0.007 mol) of anhydrous pulverized AlCl₃ inside of dry box. It was equipped with thermocouple, magnetic stir bar and dry ice condenser connected to nitrogen line. The reactor was cooled down with ice, charged with 11 g (0.042 mol) of HFO-153-10ze (C₄F₉CH═CHF) and 5 g (0.042 mol) of chlorotrifluoroethylene (CTFE) was introduced into reaction mixture through gas inlet tube over period of 30 mins or minutes. The reaction vessel was slowly warmed up to ambient temperature in water bath and was kept agitated for 4 hours. Crude reaction mixture was diluted with 300 ml of water, organic layer was separated, dried over MgSO₄ and filtered to give 15 g of crude material, which was distilled using 10 inch Vigreux column to give 7.9 g (75%) of material boiling at 120-129° C. and containing a mixture of C₄F₉CH═CHCF₂CF₂C₁ and C₄F₉CH═CHCFClCF₃ (ratio 54:46), along with 3% of higher boiling point material.

E-C₄F₉CH═CHCF₂CF₂C₁:

¹⁹F NMR (CDCl3, J, Hz): −71.53 (2F, t, 4.7, Hz), −81.10 (3Ft, 8.5, Hz), −113.68 (2F, m), −114.16 (2F, m) −124.35 (2F, m), −125.85 (2F, m) ppm

¹H NMR (CDCl3 J, Hz): 6.50 (m)

E-C₄F₉CH═CHCFClCF₃:

¹⁹F NMR (CDCl3, J, Hz): −81.10 (3Ft, 8.5, Hz), −81.84 (3F, d, 7.1, Hz), −113.68 (2F, m), −124.35 (2F, m), −125.85 (2F, m), −131.68 (1F, m) ppm

¹H NMR (CDCl3 J, Hz): 6.50 (m)

MS (z/e, mixture of isomers): 361 [(M-F)⁺, C₈H₂ClF₁₂⁺)

The reaction product ratio of C₄F₉CH═CHCF₂CF₂C₁ and C₄F₉CH═CHCFClCF₃ can range from about 64:36, about 62:38, and in some cases about 60:40.

### Example 4

Reaction of HFO-1234ze with tetrafluoroethylene catalyzed by AlCl₃

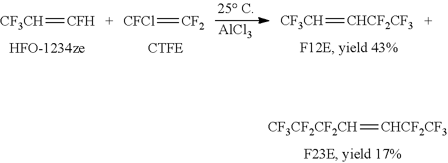

A 400 ml Hastelloy® shaker tube was loaded with 5 g (0.038 mol) of anhydrous pulverized AlCl₃, shaker tube was cooled down in dry ice, evacuated and charged with 60 g (0.52 mol) of HFO-1234ze and 50 g (0.5 mol) of tetrafluoroethylene (TFE). Shaker tube was placed in barricade and was warmed up to ambient temperature for 2 hours. It was charged with another 50 g (0.5 mol) of TFE and kept agitated for 12 hours. The reactor was cooled down with ice, vented off and liquid product (140 g) was added to 1 L of water. Organic layer was separated, dried over MgSO₄ and filtered to give 130 g of crude material, containing 65% of E-CF₃CH═CHCF₂CF₃ (F12E) and 35% E-C₂F₅CH═CHC₃F₇ (F23E). Fractionation using a 10 inch Vigreux column gave 46 g (yield 43%) of identified by GC/MS and NMR as CF₃CH═CHCF₂CF₃ (b.p. 29-30° C.) and 28 g (yield 17%) of material b.p. 70-74° C. (main 73-74° C.) identified by NMR and GC/MS as E-C₂F₅CH═CHC₃F₇ (purity 98%).

E-CF₃CH═CHCF₂CF₃:

¹⁹F NMR (CDCl₃): −66.30 (3F, dm, 4.1, 1.5 Hz), −85.07 (3F, m), −117.98 (2F, dm, 8.7, 2.3 Hz) ppm

GC/MS (m/z): 214 (M⁺, C₅H₂F₈⁺) ¹H NMR(CDCl₃): 6.46 (m) ppm

E-C₂F₅CH═CHC₃F₇:

¹⁹F NMR (CDCl₃): −80.66 (3F, t, 9.1 Hz), −85.07 (3F, m), −115.28 (2F, quint., 8.7 Hz), −117.88 (2F, dm, 8.5, 2.0 Hz), −127.88 (2F,$) ppm.

¹H NMR(CDCl3): 6.46 (m) ppm

GC/MS (m/z): 314 (M⁺, C₇H₂F₁₂⁺)

If desired, the E-CF₃CH═CHCF₂CF₃ (F12E) and E-C₂F₅CH═CHC₃F₇ (F23E) in the reaction product mixture can be varied by varying the amount of reactants. The amount of E-CF₃CH═CHCF₂CF₃ (F12E) and E-C₂F₅CH═CHC₃F₇ (F23E) in the reaction product can vary from 1 to 100 wt %, about 25 to 75 wt. % and in some cases about 50 to 50 wt. %.

### Example 5

Reaction of HFO-1234ze with vinylidene fluoride catalyzed by AlCl₃

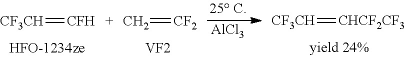

This reaction was carried out in similar fashion in 400 ml Hastelloy® shaker tube, using with 5 g (0.038 mol) of anhydrous pulverized AlCl₃, 60 g (0.52 mol) of HFO-1234ze and 32 g (0.5 mol) of vinylidene fluoride (VF2) added to cold reaction vessel in one portion. The reaction mixture was worked up as it was described above. Crude product (89 g) was distilled to give 21 g (yield 24%) of fraction with boiling point 63-68° C., identified as a mixture of E-CF₃CH═CHCH₂CF₃ and Z—CF₃CH═CHCH₂CF₃ (ratio 92:8) by GC/MS and NMR, along with 60 g of higher boiling point material, which was not characterized.

E-CF₃CH═CHCH₂CF₃:

¹⁹F NMR (CDCl₃): −65.93 (3F, t, 9.2 Hz), −65.31 (3F, dm, 5.2, 1.5 Hz) ppm

¹H NMR(CDCl₃): 2.97 (2H, quint, 8.6 Hz), 5.91 (1H, m), 6.33 (1H, m) ppm Z—CF₃CH═CHCH₂CF₃:

¹⁹F NMR (CDCl₃): −59.36 (3F, d, 7.9 Hz), −66.41 (3F, t, 9.2 Hz) ppm

¹H NMR (CDCl₃): 3.16 (2H, quint, 8.6 Hz), 5.91 (m), 6.33 (1H, m) ppm

GC/MS (m/z, mixture isomers): 178 (M⁺, C₅H₄F₆⁺)

Reaction of HFO-1234yf with tetrafluoroethylene catalyzed by AlCl₃ (Comparative Example).

A reaction of 5 g (0.038 mol) of anhydrous, pulverized AlCl₃, 115 g (1 mol) of HFO1234yf (CF₃CF═CH₂, isomer of HFO-1234ze) and 50 g of TFE was carried out as described above in 400 ml Hastelloy® shaker tube at ambient temperature. No pressure drop was observed over a 16-hour period and no liquid product was recovered after shaker tube was vent off

The ratio of E-CF₃CH═CHCH₂CF₃ and Z—CF₃CH═CHCH₂CF₃ in the product mixture can range from about 1 to 100 wt %, about 25 to 75 wt. % and in some cases about 50 to 50 wt %. The ratio can be varied by changing at least one of the ratio of reactants, an optional solvent and temperature.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A method of producing a fluoroolefin comprising:
contacting a compound of formula (1),
R_{f}CH═CHF (1)
wherein R_{f} is a C₁-C₁₀ perfluorinated or polyfluorinated alkyl group;
with a fluorinated ethylene compound of formula (2),
CX₁X₂═CX₃X₄ (2)
wherein X₁, X₂, X₃, and X₄ are each independently H, Cl, or F; and
wherein at least one of X₁, X₂, X₃, and X₄ is F;
in the presence of a Lewis acid catalyst in an amount sufficient to form a composition comprising a compound of formula (3),
R_{f}CF₃(CX₅X₆CX₇X₈)ₙCH═CHCX₉X₁₀CX₁₁X₁₂F (3)
wherein X₅, X₆, X₇, X₈, X₉, X₁₀, X₁₁, and X₁₂ are each independently H, Cl, or F, n is an integer of 0 or 1; and
wherein the total number of each of H, Cl, and F represented by X₅, X₆, X₇, X₈, X₉, X₁₀, X₁₁, and X₁₂ is the same as the total number of each of H, Cl, and F provided by the fluorinated ethylene compound of formula (2).

2. The method of claim 1 wherein:
the compound of formula (1) includes CF₃CH═CHF (1234ze); and
the composition includes 1,1,1,4,4,5,5,5-octafluoropent-2-ene, CF₃CH═CHC₂F₅ (F12E).

3. The method of claim 1 wherein:
the fluorinated ethylene of formula (2) includes CF₂═CF₂ (TFE); and
the composition includes 1,1,1,4,4,5,5,5-octafluoropent-2-ene, CF₃CH═CHC₂F₅ (F12E).

4. The method of claim 1 wherein:
the compound of formula (1) includes CF₃CH═CHF (1234ze); and
the fluorinated ethylene of formula (2) includes CF₂═CF₂ (TFE); and
the composition includes 1,1,1,4,4,5,5,5-octafluoropent-2-ene, CF₃CH═CHC₂F₅ (F12E).

5. The method of claim 4, wherein the amount sufficient includes a molar ratio of (TFE):(1234ze) of 0.01:1 to 5:1.

6. The method of claim 5, wherein the amount sufficient includes a molar ratio of (TFE):(1234ze) of 0.1:1 to 2:1.

7. The method of claim 1, wherein the amount sufficient includes a molar ration of the compound of formula (2) and the compound of formula (1) of 0.01:1 to 5:1.

8. The method of claim 1, wherein the composition further comprises 1,1,1,2,2,5,5,6,6,7,7,7-dodecafluorohept-2-ene, C₃F₇CH═CHC₂F₅ (F23E).

9. The method of claim 1, wherein the composition further comprises 4-chloro-1,1,1,4,5,5,5-heptafluoropent-2-ene CF₃CH═CHCFClCF₃ and 5-chloro-1,1,1,4,4,5,5-heptafluoropent-2-ene CF₃CH═CHCF₂CF₂C₁.

10. The method of claim 1, wherein the composition further includes at least one of diluents and solvents.

11. The method of claim 10, wherein the solvent is a perfluorinated saturated compound.

12. The method of claim 11, wherein the perfluorinated saturated compound is selected from the group insisting of perfluoropentane, perfluorohexane, cyclic dimer of hexafluoropropene, (mixture of perfluoro-1,2- and perfluoro-1,3-dimethylcyclobutanes), and combinations thereof

13. The method of claim 1 wherein:
the fluorinated ethylene of formula (2) includes CClF═CF₂ (CTFE); and
the composition includes 4-chloro-1,1,1,4,5,5,5-heptafluoropent-2-ene, CF₃CH═CHCClFCF₃ or 5-chloro-1,1,1,4,4,5,5-heptafluoropent-2-ene, CF₃CH═CHCF₂CClF₂.

14. The method of claim 13 wherein:
the catalyst includes aluminum chloride (AlCl₃) or a compound of formula (4),
AlClₓF₃₋ₓ
wherein x=0.01 to 0.5.

15. The method of claim 14, wherein the catalyst includes aluminum chloride (AlCl₃).

16. The method of claim 1, wherein R_{f} is a C₂-C₁₀ perfluorinated alkyl group.

17. The method of claim 1, wherein R_{f} is CF₃, C₂F₅, C₃F₇, iC₃F₇, C₄F₉, C₅F₁₁, i-C₅F₁₁, C₆F₁₃ or i-C₆F₁₃.

18. The method of claim 1, wherein the contacting is performed at sub-ambient or ambient temperature.

19. The method of claim 1, wherein the reaction is conducted under autogenic pressure.

20. The method of claim 1, wherein the reaction is conducted at 0.1 to 300 psig.

21. The method of claim 1, wherein the reaction is conducted in a closed system.

22. The method of claim 1, wherein the contacting is performed at a temperature of −50° C. to 50° C.

23. The method of claim 1, wherein the catalyst includes aluminum chloride (AlCl₃) or aluminum chlorofluoride AlClₓF₃₋ₓ (ACF) wherein x=0.01 to 0.5.

24. The method of claim 23, wherein the catalyst includes aluminum chloride (AlCl₃).

25. The method of claim 10 wherein the at least of diluents and solvents comprises a reaction product formed by said contacting.

26. A method of producing a fluoroolefin comprising:
contacting CF₃CH═CHF (1234ze) with CF₂═CF₂ (TFE) in the presence of a catalyst in an amount sufficient to form a composition comprising 1,1,1,2,2,5,5,6,6,7,7,7-dodecafluorohept-2-ene, C₃F₇CH═CHC₂F₅ (F23E).

27. The method of claim 26, wherein the contacting is performed at a temperature of −10° C. to 50° C.

28. The method of claim 26, wherein the composition further comprises 1,1,1,4,4,5,5,5-octafluoropent-2-ene, CF₃CH═CHC₂F₅ (F12E).

29. The method of claim 26, wherein the composition further comprises 4-chloro-1,1,1,4,5,5,5-heptafluoropent-2-ene, CF₃CH═CHCFClCF₃ and 5-chloro-1,1,1,4,4,5,5-heptafluoropent-2-ene, CF₃CH═CHCF₂CF₂C₁.

30. The method of claim 26, wherein the composition further includes at least one of diluents and solvents.

31. The method of claim 30, wherein the solvent is a perfluorinated saturated compound.

32. The method of claim 31, wherein the perfluorinated saturated compound is selected from the group insisting of perfluoropentane, perfluorohexane, cyclic dimer of hexafluoropropene, (mixture of perfluoro-1,2- and perfluoro-1,3-dimethylcyclobutanes), and combinations thereof

33. The method of claim 26, wherein the amount sufficient includes a molar ratio of (TFE):(1234ze) of 0.01:1 to 5:1.

34. The method of claim 33, wherein the amount sufficient includes a molar ratio of (TFE):(1234ze) of 0.1:1 to 2:1.

35. The method of claim 26, wherein the contacting is performed at sub-ambient or ambient temperature.

36. The method of claim 26, wherein the reaction is conducted under autogenic pressure.

37. The method of claim 26, wherein the reaction is conducted at 0.1 to 300 psig.

38. The method of claim 26, wherein the reaction is conducted in a closed system.

39. The method of claim 26, wherein the catalyst includes aluminum chloride (AlCl₃).

40. The method of claim 30 wherein the at least one diluent and solvent comprises a reaction product formed by said contacting.

41. A composition formed by the method of claim 1.

42. A composition formed by the method of claim 2.

43. A composition formed by the method of claim 4.

44. A composition formed by the method of claim 26.

45. A composition formed by the method of claim 28.

46. A method of producing a fluorooligomer comprising:
heating CF₃CH═CHF (1234ze), in the presence of a catalyst, at a temperature and a pressure sufficient to form a composition comprising CF₃CH═CHCH(CF₃)CF₂H.

47. The method of claim 44, wherein the catalyst includes aluminum chloride (AlCl₃) or aluminum chlorofluoride AlClₓF₃₋ₓ (ACF) wherein x=0.01 to 0.5.

48. A composition formed by the method of claim 44.
